(19) United States
(12) Patent Application Publication
(10) Pub. No.: US 2004/0135971 A1
(43) Pub. Date: Jul. 15, 2004

(54) Device for viewing an eye

(75) Inventor: Gerd Ulbers, Kehrsatz (CH)

Correspondence Address: Matthew R. Jenkins, JACOX, MECKSTROTH & JENKINS, Suite 2, 2310 Far Hills Building, Dayton, OH, 45419-1575, US

(73) Assignee: HAAG-STREIT AG

(21) Appl. No.: 10/702,294

(22) Filed: Nov. 6, 2003

(30) Foreign Application Priority Data
Nov. 7, 2002 (EP) 02 405 961.0

(51) Int. Cl. A61B 3/10

(52) U.S. Cl. 351/221

(57) ABSTRACT

The inventive slit-lamp unit comprises a table (1), a cross-slide (2) which can be displaced with the aid of a guide lever (8), a head holder (3), a microscope (5) which can be rotated about an axis of rotation (9), and an illuminating means (10) fastened on a holder (11). The holder (11) comprises a base (11.1), likewise rotatable about the axis of rotation (9), and a carrier (11.2) which can be displaced relative to the base (11.1) by means of a rail (13) in the direction of the eye (7) or away from the latter. The illuminating means (10) are fastened on the carrier (11.2) and comprise a lens (19), a prism (18), an image recording device (14) and a light source (17). The prism (18) serves the purpose both of deflecting onto the eye (7) an illuminating beam generated by the light source (17), and of deflecting onto the image recording device (14) a viewing beam emerging from the eye (7), the said image recording device being connected to a computer monitor (16.2) in order to display the images thus recorded. If the illuminating means (10) are positioned directly in front of the eye (7) with the aid of the rail (13), it is also possible to use this slit-lamp unit for high-quality examination of the fundus of the eye.

### TECHNICAL FIELD

The invention relates to a device for viewing an eye of a patient, in particular a slit-lamp unit, comprising viewing means for viewing a front section of the eye and illuminating means for illuminating the front section of the eye.

### PRIOR ART

There are many different apparatuses and devices for optometric examination, diagnosis and/or treatment. It is frequently possible in this case to use a specific unit only for one type of examination, since, for example, the examination of the front eye section (cornea etc.) differs fundamentally from the examination of the rear eye section (fundus). The reason for this, inter alia, is because during examination of the fundus the latter must be illuminated through the pupil so that it is possible to detect anything at all. By examining the front eye section, by contrast, it is possible in principle for the areas to be examined to be illuminated using arbitrary means and from any desired sides. The front eye section can be viewed even without artificial illumination. The multiplicity of types of units is due, furthermore, to the fact that other imaging properties are possible, desired or required depending on the areas of the eye to be examined.

So-called slit-lamp units such as are described, for example, in WO 99/23937 A1 are typically used to view the front eye sections. Such slit-lamp units generally comprise a microscope and an illuminating device, which are mounted on a unit pedestal such that they can be displaced jointly, and are supported independently of one another such that they can rotate about a common, vertically situated axis of rotation. The light from the illuminating device is directed via a deflecting mirror onto the eye, which can be viewed using the microscope.

Special units, so called ophthalmoscopes, are likewise typically used for viewing the rear eye sections. These occur in various embodiments, from the simple ophthalmoscope to the highly complex fundus camera.

However, there is a common problem with both types of units, slit-lamp units and ophthalmoscopes: since they are designed for viewing the front or rear eye sections, they are respectively unsuitable or only slightly suitable for viewing the respective other eye section.

Thus, for example, it is impossible to view the fundus using a slit-lamp unit. Specifically, this would require ability to position the microscope directly in front of the eye, something which is impossible because of the described properties of the microscope or which can be varied only in narrow limits, and because of the illumination.

A solution to this problem consists, for example, in using additional optical aids which are inserted into the beam path. Thus, for example, WO 01/87146 describes how the fundus of the eye can be viewed with the aid of a slit-lamp unit and an auxiliary lens inserted into the beam path between the eye to be examined and the microscope. However, only a relatively poor imaging quality can be achieved with the aid of this viewing technique, since it gives both at the cornea and at the auxiliary lens to undesired optical reflections which complicate the viewing of the fundus. In addition, the positioning of the auxiliary lens in front of the eye must either be performed by hand, something which is very difficult even for an expert, or it is performed with the aid of the microscope, the auxiliary lens thereafter needing to be decoupled from the microscope for the purpose of viewing the fundus, as described in the abovenamed publication.

### SUMMARY OF THE INVENTION

It is therefore an object of the invention to specify a device of the type mentioned at the beginning which permits with the aid of a single unit both the front and the rear eye sections to be viewed with high quality.

How the object is achieved is defined by the features of Claim 1.

The inventive device is a unit for viewing the front eye sections, in particular a slit-lamp unit which is modified in such a way that it can be used to view the fundus of the eye without reflections and with high quality. The device comprises viewing means for viewing a front eye section, the viewing means comprising, in particular, the microscope, for example a Greenough microscope, fastened on a holder. The device further comprises illuminating means for illuminating the front eye sections, the illuminating means having a light source whose light is directed onto the eye directly or indirectly.

As is known with slit-lamp units, the viewing means and the illuminating means are advantageously arranged in such a way that they can be pivoted independently of one another about a common, vertical axis of rotation. Were they mounted rigidly, it would be possible to examine in each case only a small area of the eye during the various eye examinations. The inventive slit-lamp unit also comprises fixing means, in particular. These serve the purpose of fixing the patient's head, and thus the eye to be examined, in space, in order to avoid as far as possible undesired movements of the patient during an examination. It would be extremely difficult for a patient without such a head holder to sit motionless for minutes on end.

By contrast with the slit-lamp units known from the prior art, the inventive device is also suitable for viewing the fundus of the eye, since, on the one hand, the illuminating means are designed in such a way that they can be used to illuminate not only the front eye sections such as, for example, the cornea, but also a rear eye section such as, for example, the fundus. On the other hand, the inventive device has an image acquisition device for acquiring images of the eye which has an image recording device which is designed and can be positioned in such a way that the rear eye section can be imaged ophthalmoscopically thereby. That is to say, the rear eye sections can be viewed and imaged with the aid of this additional image acquisition device and not, or not exclusively, with the aid of the viewing means with the aid of which the front eye sections are viewed.

For the purpose of correctly imaging the rear eye areas to be examined, the device additionally comprises optical imaging means such as, for example, one or more lenses, which is or are positioned between the eye and the image recording device.

Since the fundus is viewed with the aid of an image acquisition device designed therefor, the need to vary the optical properties of the viewing means used to view the front eye sections is eliminated. This image acquisition device is now designed in such a way that it, or specific parts thereof, can be positioned as near as possible in front of the eye for ophthalmoscopic viewing of the fundus of the eye. It thereby becomes possible for large areas of the fundus of the eye to be imaged and viewed with a quality hitherto absent with the aid of a slit-lamp unit. Moreover, it is even possible for further, if also smaller, areas of the eye to be viewed simultaneously with the aid of the microscope used for viewing the front eye sections.

It is likewise possible to design the image recording device in such a way that a plurality of partial images of the fundus of the eye can simultaneously be acquired and combined in such a way that the fundus of the eye can be viewed stereoscopically.

As already mentioned further above, the illuminating means comprise a light source for generating an illuminating beam. The latter is cast directly or indirectly onto or through the pupil in the eye. However, different illuminations are typically required or desired in order to illuminate the front and rear eye sections. In order to obtain the best possible illumination for the examination to be carried out, it would be possible for the illuminating means therefore to be of modular design, for example, so that a different light source or a different optical system could be used depending on the desired illumination, for example.

However, it is more advantageous when the illuminating means are designed in such a way that the type of illumination can be switched over simply from a foreground illumination to a background illumination. It should be possible to carry this out with the fewest possible manipulations, preferably by pressing a button or by throwing a lever etc.

For example, the image acquisition device could be designed in such a way that it is inserted directly into the beam path, that is to say that the image recording device, for example a camera, is positioned at the correct distance directly in front of the eye. However, it is difficult in this case to illuminate the fundus of the eye correctly and adequately.

The image acquisition device is therefore preferably designed in such a way that it comprises a deflecting device in addition to an image recording device. In order to be able to image the fundus of the eye ophthalmoscopically, it is only necessary in this case to position the deflecting device at the desired distance close in front of the eye. The deflecting device can be fabricated to be small enough not to substantially complicate the illumination of the eye. The image recording device need not then be arranged directly in front of the eye, but can, for example, be arranged laterally offset and, optionally, also further removed. The deflecting device, which is positioned in front of the eye, deflects onto the image recording device a viewing beam which emerges from the eye and strikes the deflecting device.

The deflecting device is correspondingly designed in such a way that it reflects light of any desired wavelength region more or less strongly in a specific direction. Depending on the reflection desired or required, or on other optical properties, a mirror or a prism, for example, can be used for the deflection. The use of a prism for coupling out or deflecting the viewing beam is preferred, however, since a prism typically has fewer light losses.

Of course, it is possible to design the image acquisition device as a separate unit which can be introduced into the observing beam path in a simple way when required, for example swiveled in. However, this requires a lot of space and complicates the manipulation of the slit-lamp unit. In order to save space and material, and to ensure manipulation which is as simple as possible, the inventive device is advantageously designed in such a way that the illuminating means and the image acquisition device form a unit. That is to say, the image acquisition device is, as it were, integrated into the existing illuminating means, or vice versa. The device is designed, in particular, in such a way that the illuminating beam generated by the light source is deflected onto the eye by the same deflecting device which is also used to deflect onto the image recording device a viewing beam emerging from the eye.

The light source for generating the illuminating beam is generally not positioned directly in front of the eye to be examined in the case of a slit-lamp unit, since in this case it would disturb the viewing of the eye with the aid of the viewing means. The illuminating beam is typically likewise cast onto the eye via a mirror. Here, as well, a prism, for example, can be used instead of the mirror. The illumination can then be designed in such a way that the light source cannot be displaced relative to the mirror. That is to say, the angle at which the illuminating beam is cast onto the eye would be invariable in this case. The illuminating means are preferably, however, designed in such a way that this angle is variable, in order to avoid as far as possible undesired and disturbing optical reflections of the illuminating beam. The light source is, for example, arranged such that it can swivel about a horizontal swiveling axis and can thus be swiveled relative to the mirror. For example, if the light source is located in the upper region of the slit-lamp unit and generates an illuminating beam directed substantially vertically downwards onto the mirror, the angle at which the illuminating beam strikes the eye can be varied by swiveling the light source.

The illuminating means can be implemented in the most varied way, it being possible to use various light sources such as thermal light sources, gas discharge light sources or laser light sources. Infrared light can also be used, for example. Consequently, the patient whose eye is to be examined is subjected to less glare, and the eye adapts to the darkness in the case of dark surroundings, that is to say the pupil of the eye to be examined is opened wide.

The light required can, of course, also be generated at the most varied locations, both directly from where it is radiated onto the eye, and at another location, it being guided in this case, for example, to the desired location of use by means of optical conductors.

In a preferred embodiment of the invention, the illuminating means are designed in such a way that the illuminating beam generated for illuminating the rear eye section comprises a plurality of annularly arranged individual beams directed onto the eye. Here, as well, it holds that the individual beams are generated either directly by annularly arranged light sources such as, for example, light-emitting diodes, and directed onto the eye, or that the individual illuminating beams are generated by one or more remotely situated light sources and directed onto the eye, for example, by means of optical conductors whose ends are annularly arranged. Of course, the annular illumination can also be generated by appropriately shaping the light from a light source. For example, it would be possible to insert into the illuminating beam path an annular diaphragm which blocks out the light from the light source in the center and passes it only in an annulus about the center. In order to influence the examination of the eye as little as possible, the illuminating beams are arranged annularly about the viewing beam.

There are various possibilities for designing the slit-lamp unit so that the image recording device can be used for ophthalmoscopic imaging of the fundus of the eye. If, as mentioned further above, the image acquisition device and the illuminating means form, for example, a unit, this unit must be positioned as close as possible in front of the eye so as to render ophthalmoscopic imaging of the fundus of the eye possible at all.

However, this is not possible in the case of conventional slit-lamp units, since they have a mechanical axis of rotation or shaft in the region of the patient's head about which both the microscope and the illuminating means are swiveled in a circular fashion. However, it is precisely these mechanical axes or shafts in the region directly in front of the patient which preclude positioning the image acquisition device directly in front of the eye because upon being displaced in the direction of the patient they would bump against the latter's body or head before they are positioned close enough in front of the eye to be able to view the fundus ophthalmoscopically. One possibility for solving this problem consists in designing the holder of the illumination in such a way that the mechanical axis or shaft is not situated in the region directly in front of the patient, but above or below the patient's head. However, this entails a slit-lamp unit which has substantially larger dimensions than conventional devices. Consequently, undesired instabilities would thereby arise in certain circumstances when manipulating the device.

A further possibility therefore consists in implementing the axis of rotation about which the illuminating means and the microscope can be swiveled not by means of mechanical axes or shafts but by means of rail-type guide means. That is to say, the illuminating means are connected to the device pedestal via rail-type guide means, for example. The illuminating means can then be rotated about the vertical axis of rotation by swiveling along these rail-type guide means. The microscope could also be connected to the device pedestal in the same way via rail-type guide means. Positioning the image acquisition device would then require merely to displace the device pedestal together with the microscope and illumination in a direction of the patient. Since no mechanical axis or shaft is any longer present in the region in front of the patient, the device pedestal can be positioned without hindrance close enough in front of the eye to be examined.

In one preferred possibility, however, the deflecting device and the image recording device are fastened on a holder which comprises a base and a carrier. As is known from the prior art, the base, of elongated design and lying horizontally, for example, is connected to the slit-lamp unit such that it can rotate about a vertical, mechanical axis of rotation or shaft. By contrast with the known slit-lamp units, in the case of which the illuminating means are connected rigidly to the base, the elongated, vertically arranged carrier is connected here to the base via guide means. Consequently, the carrier, at the upper end of which the deflecting device is arranged, can be displaced relative to the base.

The guide means are preferably designed in this case in a rail-type fashion such that the carrier can be displaced with the deflecting device in a translatory fashion along the base in the direction of the eye or away therefrom. Here, the vertical axis of rotation about which the deflecting device can be swiveled is retained. That is to say, the deflecting device can be rotated about the vertical axis of rotation even if it is positioned extremely close in front of the eye. Thus, if the device pedestal of the inventive slit-lamp unit is positioned in such a way that the vertical axis of rotation runs in the region of the eye, for example approximately in the pupil plane, various areas of the fundus of the eye can be examined through the pupil by rotating the carrier with the deflecting device.

Any desired image recording device can be used in principle in order to record the images, it being preferred to use analog photographic or video cameras or digital cameras with a CCD (charge coupled device) or a CMOS (complementary metal oxide semiconductor) image chip.

The recorded images are then present in a format dependent on the respective camera. If, for example, a photographic camera with a light-sensitive film strip is used, the pictures are present as negative or positive after the development of the film strip. In conventional, analog video cameras with magnetic tapes, the image information is stored as an analog, magnetic pattern on the tapes. If, by contrast, digital cameras such as, for example a digital photographic or video camera are used, it is not film strips which are exposed, but light-sensitive chips with a multiplicity of pixels. In the case of such chips, the acquired images can be further processed in a directly electronic fashion. Suitable for this purpose are, for example, CCD chips or so called CMOS chips, the latter having the advantage that a higher picture frequency is achieved and the electronically acquired images can be further processed immediately with the aid of a CMOS logic system accommodated on the same chip.

The images can be stored, for example, in an optional digital image format on any desired electronic storage medium. However, they can also be displayed directly on a screen, for example an LCD screen of the camera itself or on a computer monitor as individual images or else as image sequences with an optional picture frequency. The type of image display is arbitrary, and ranges from the normal television set with an appropriate adapter via the already mentioned computer monitor to projectors which project the electronically available images onto a wall or projection screen.

The image acquisition device correspondingly preferably also comprises an image displaying device for displaying the pictures of the fundus of the eye. The display can be performed both in real time during the examination of the eye, and at a later point in time, the pictures then simply being read out again from the memory used.

The optical imaging means mentioned which are used to image the fundus of the eye can be provided both near the eye, that is to say in the vicinity of the eye, as well as near the camera, that is to say in the vicinity of the image recording device, preference being given to the positioning near the eye.

Of course, the inventive device can also have further components. Thus, the image acquisition device could be designed, in such a way that only a (prescribed) magnification is possible when imaging the fundus. However, the image acquisition device is preferably designed in such a way that, for example, it can be switched over between a plurality of, that is to say at least two imaging magnifications by means of a change in optics. That is to say, a variable magnification can be achieved with the aid of one or more additional lens systems which can be switched between the eye and the image recording device.

Further advantageous embodiments and combinations of features of the invention emerge from the following detailed description and the totality of the patent claims.

### BRIEF DESCRIPTION OF THE DRAWINGS

In the drawings used to explain the exemplary embodiment,

FIG. 1 shows a diagrammatic illustration of an inventive device for viewing the front and/or rear eye sections;

FIG. 2 shows a diagrammatic illustration of the table with the pedestal plate of the device from FIG. 1, from above;

FIG. 3 shows a diagrammatic illustration of the scale ratios with the device from FIG. 1;

FIG. 4 shows a diagrammatic illustration of an illumination variant for the device from FIG. 1;

FIG. 5 shows a diagrammatic illustration of a further illumination variant for the device from FIG. 1;

FIG. 6 shows a diagrammatic illustration of a prism for a further illumination variant, from the side;

FIG. 7 shows a prism from FIG. 6, from the front, that is to say seen from the eye to be examined;

FIG. 8 shows a diagrammatic illustration of another exemplary embodiment of the invention;

FIG. 9 shows a diagrammatic illustration of the table with the pedestal plate of the device from FIG. 6, from above;

FIG. 10 shows a diagrammatic illustration of a deflecting device, designed as a mirror, from the side, in a position for viewing the fundus of the eye;

FIG. 11 shows the deflecting device from FIG. 8, from the front and

FIG. 12 shows the deflecting device from FIG. 8, from the side, in the position for viewing the anterior of the eye.

Identical parts are fundamentally provided with identical reference numerals in the figures.

### WAYS OF IMPLEMENTING THE INVENTION

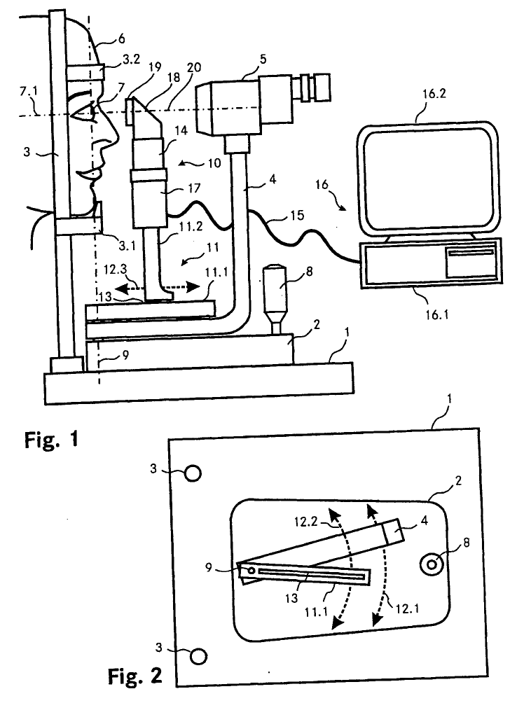

FIG. 1 shows an inventive device in a schematic illustration, from the side. The device comprises a table 1, a cross-slide 2, a head holder 3 with a chin support 3.1 and a forehead support 3.2, a microscope 5 mounted on a microscope arm 4, and illuminating means 10 which are fastened on a holder 11. The microscope arm 4 and the holder 11 are connected to the cross-slide 2 in such a way that they can be swiveled in a circular arc about a common, geometrical, vertically situated axis of rotation 9. Provided at the location of the axis of rotation 9 is, for example, a mechanical axis on which the microscope arm 4 and the holder 11, which are provided with appropriate cutouts, can be mounted. Fixed in the head holder 3 is the head 6 of a patient whose right eye 7 is to be examined with the aid of the microscope 5.

FIG. 2 shows the table 1 and the cross-slide 2 from above, the vertical axis of rotation 9 being represented merely as a point in this view. The rotary movements of the microscope arm 4 and the base 11.1 of the holder 11 are illustrated by the arrows 12.1 and 12.2 respectively, drawn with dashes.

As is known from the prior art, together with the illuminating means 10 and microscope 5 it Is possible for the cross-slide 2 to be displaced with the aid of the guide lever 8 horizontally on the table 1 (for example by inclining the guide lever 8) and vertically (for example by rotating the guide lever 8). The guide lever 8 can be used to position the cross-slide 2 in front of the head 6 of the patient in such a way that the left eye (not visible) can also be examined with the aid of the microscope 5.

The inventive device has at least two essential differences from the slit-lamp units known from the prior art. On the one hand, otherwise than in the prior art, the holder 11 for the illuminating means 10 is not of unipartite design, but is bipartite, the upper part of the holder 11, the carrier 11.2, being supported with the illuminating means 10 such that it can be displaced relative to the lower part of the holder 11, the base 11.1. On the other hand, the illuminating means 10 comprise not only the means for illuminating the eye 7, but also the image recording device 14 for recording images of the eye 7.

Furthermore, the illuminating means 10 comprise a light source 17 for generating the illuminating beams, and a prism 18 for deflecting the generated illuminating beams onto the eye 7. In addition, there is fastened on the prism 18 a lens 19, which is required for the correct imaging of the fundus of the eye. In order to be able to adapt the type of illumination to the examinations to be carried out, the illumination means 10 further has means (not illustrated), for example for producing various cross sections of the illuminating beam such as annular or slits as well as, for example, filters or different light sources.

An image recorded by the image recording device 14 is present, for example, electronically in the form of pixel values. In the example illustrated in FIG. 1, such an image is transmitted via a cable 15 to a computer 16 with an arithmetic unit 16.1 and a monitor 16.2, and can either be displayed directly on the monitor 16.2, or be stored in a memory of the arithmetic unit 16.1. A display of an image or an image sequence can either be performed in real time, or it can be performed at a later time with the aid of the stored images.

Otherwise than in the case of the prior art, the holder 11 of the illuminating means is not of L-shaped design like the microscope arm 4 but, as already mentioned, comprises a base 11.1 and a carrier 11.2. The base 11.1 is of elongated design and can be rotated in a horizontal plane about the axis of rotation 9. The base 11.1 has a rail 13 on its top side. The carrier 11.2, which is likewise of elongated design, but is fastened in a substantially vertical fashion, has a counterpart to the rail 13 at its lower end. The carrier 11.2 can, for example, be displaced by means of a roller or slide bearing along the rail 13 in relation to the base 11.1, that is to say in the direction of the axis of rotation 9, or away from the latter in the direction of the arrow 12.3. The illuminating means 10 and the image recording device 14 are fastened at the upper end of the carrier 11.2.

Once the cross-slide 2 is positioned in front of the eye 7 to be examined, the carrier 11.2 can be brought directly in front of the eye 7 by displacement on the base 11.1 together with the illuminating means 10 and the image recording device 14. The distance of the lens 19 from the eye 7 can thereby be set as desired from a few millimeters to a few centimeters. The distance is approximately 10 to 15 mm, for example, for the purpose of viewing the fundus of the eye. If, moreover, the cross-slide 2 is positioned in front of the eye in such a way that the vertical axis of rotation 9 is situated in the region of the eye 7, for example in its pupil plane, the illuminating means 10 and the image recording device 14 can respectively be swiveled in a circular fashion about the eye 7 for each selected distance. Different areas of the fundus of the eye can thereby respectively be illuminated and examined through the pupil of the eye 7.

In order to examine the eye 7, the device can be positioned in front of the eye in such a way that the optical axes 7.1 of the eye 7 and the lens 19 substantially coincide. Furthermore, the device is designed in such a way that the optical axis of the microscope 5 also coincides with the optical axes 7.1 of the lens 19 and the eye 7. Consequently, given an appropriate construction of the prism 18 from (partially) transparent material, it is also possible to examine the eye 7 both with the aid of the image recording device 14 and, at the same time, with the aid of the microscope 5.

It may be mentioned as a supplement at this point that additional means may be present for fixing the holder 11 and the microscope arm 4 on the cross-slide 2. However, for reasons of clarity such means have not been illustrated. The same holds for the means for limiting the translation movement of the carrier 11.2 on the base 11.1.

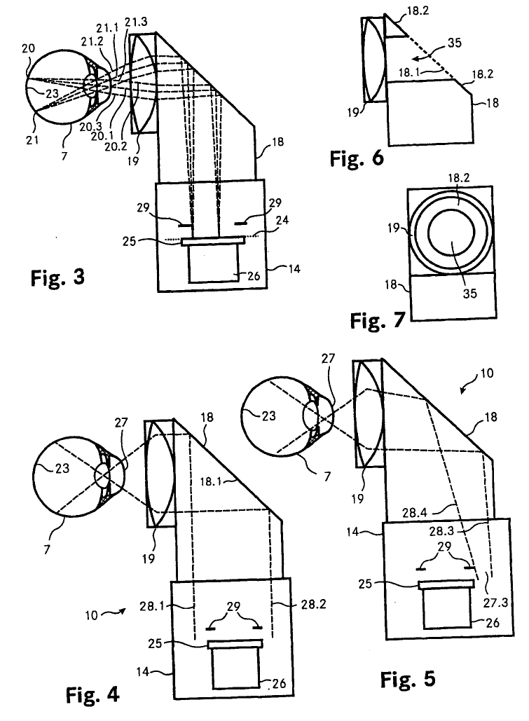

FIGS. 3 to 5 show diagrams of the beam paths inside the illuminating means 10.

FIG. 3 shows, in particular, the beam path for the imaging of the fundus 23 of the eye 7. Only the beams for two fundus points 20 and 21 of the fundus 23 of the eye are illustrated as representative of the entire viewing beam. Starting from the fundus 20, three beams 20.1, 20.2, 20.3 are illustrated, the beam 20.1 being the middle beam, that is to say the beam through the center of the lens 19. The two beams 20.2 and 20.3 are those beams which are just still capable of leaving the eye 7 through the pupillary aperture at the top and bottom, respectively, starting from the fundus point 20. The same holds for the fundus point 21 with the middle beam 21.1 and the lateral beams 21.2 and 21.3. If the eye is accommodated to infinity, the beams 20.1, 20.2, 20.3 of the fundus point 20 run parallel outside the eye 7. The same also holds for the beams emanating from the fundus point 21.

The lens 19 focuses the beams of a fundus point onto a virtual image plane. By inserting the prism 18 into the beam path, the beams are, however, deflected in such a way as to produce an image of the fundus 23 of the eye in an image plane 24. A CCD chip 25 of the image recording device 14 is arranged in this image plane 24 such that the image of the fundus 23 of the eye can thereby be recorded. Reading the image out and processing it further are performed, however, by means of an electronic system 26 which is likewise present in the image recording device 14. The cable 15, illustrated in FIG. 1, for transmitting the image data to a computer would be, for example, connected to this electronic system 26 (not illustrated).

An annular diaphragm 29 of appropriate design is inserted into the beam path in order to minimize as far as possible undesired extraneous light influences such as, for example, optical reflections falling onto the CCD chip 25. Such optical reflections are produced, for example, at the inner or outer boundary surfaces of the prism 18, the lens 19 or the eye 7.

The distance of the CCD chip 25 from the eye 7 or the lens 19 can be set by displacing the carrier 11.2. For even more precise positioning, the device could comprise additional means (not illustrated) for fine adjustment of the distance between the CCD chip 25 and eye 7. This fine adjustment can be achieved either by a horizontal displacement of the carrier 11.2 relative to the base point 1.1 or by an appropriate vertical displacement of the CCD chip 25.

FIG. 4 shows a possibility for illuminating the fundus 23 of the eye with the aid of annular illumination. The light source itself, which is positioned in the lower part of the illuminating means 10, is not illustrated. However, the light source generates a plurality of light bundles arranged annularly around the CCD chip 25. The generation of these light bundles is performed, for example, with the aid of appropriate diaphragms, or the light generated by the light source is shaped in the desired way by means of lenses and mirrors or other optical components such that the required illumination of the fundus of the eye is achieved.

One beam 28.1, 28.2 is drawn in respectively for two of these light bundles. The beams 28.1, 28.2 are deflected by the prism 18 and cast through the lens 19 onto the eye 7, the illuminating means 10 being designed in such a way that the annularly arranged beams 28.1, 28.2 are imaged annularly onto or a little in front of or behind the cornea 27 of the eye 7. As a result, no disturbing reflections are produced in the region of the cornea 27, and as large a portion as possible of the light bundles passes through the pupillary aperture of the eye 7 onto the fundus 23 of the eye.

A further possibility for illuminating the fundus 23 of the eye is illustrated in FIG. 5. Here, the illumination is not performed annularly, but comprises only a laterally incident bundle, emanating from the light source (not illustrated), of beams of which the bounding beams 28.3, 28.4 are illustrated. These are deflected in turn onto the fundus 23 of the eye 7 by a prism 18 via the lens 19 and pupil. It holds here as well, that the beams 28.3, 28.4 are imaged onto or a little in front of or behind the cornea 27 of the eye 7. Undesired reflections which disturb the investigation of the eye can be avoided, in addition, with the aid of the diaphragm 29 by coupling the illuminating beam in laterally in conjunction with an adjustable angle.

The light source which generates such a laterally incident illuminating beam need not necessarily be integrated in the illuminating means 10 in this case. It can also be provided outside the illuminating means 10, the generated illuminating beam either being directed onto the eye 7 directly from the side, or else being directed laterally in a similar way to that illustrated in FIG. 5 into the prism 18 and being directed from the latter onto the eye 7.

The illumination can also be implemented in a way known per se. The light source is located in this case in the upper region of the device and generates a light bundle directed downwards. The light source is seated on a holder which itself is fastened on a carrier in such a way that the holder together with the light source can be swiveled about a horizontal axis of rotation. Located at the upper end of the carrier is a mirror which deflects onto the eye the light bundle generated by the light source and directed downwards. That is to say, the light bundle strikes a mirror approximately at an angle of 45 degrees, and is deflected therefrom onto the eye more or less horizontally. The angle at which the light bundle strikes the eye can be varied by slightly swiveling the holder with the light source about the horizontal axis of rotation. It would also be possible in the case of a slit-lamp unit of such design for the carrier to be arranged in a horizontally displaceable fashion in the inventive way such that the holder together with the light source could be positioned close in front of the eye. Furthermore, a camera could also thereby be integrated in the illumination, in order to deflect the light emerging from the eye onto the camera via the mirror fastened at the upper end of the carrier.

FIGS. 6 and 7 show a further example of how an annular illumination of the fundus can be achieved. By contrast with the prism 18 from FIG. 4, in which the entire deflecting surface 18.1 reflects light, the prism 18 illustrated in cross section in FIG. 6 and from the front in FIG. 7 reflects light only in an annular region 18.2 of the deflecting surface 18.1 illustrated by dashes. Consequently, when the prism 18 is illuminated from below an annular illumination of the fundus 23 of the eye is likewise produced.

The annularly reflected region 18.2 could also be produced by constructing the deflecting surface 18.1 in a reflecting fashion, for example by aluminizing it, only in an appropriate region. However, as illustrated, it is also possible simply to remove the non-reflecting region of the prism. Seen from the eye 7, this produces in the prism 18 a horizontally situated hole 35 directly behind the lens 19, as it were. Through this hole 35, beams which emerge from the eye 7 and run through the central region of the lens 19 can pass unhindered through the prism 18 through a microscope (not illustrated) situated therebehind, of a further camera, with the aid of which microscope or camera it is likewise possible to view the fundus in this way. The prism 18 with the hole 35 acts in this case simultaneously as a diaphragm which blocks out undesired reflections from the region of the eye 7 or the lens 19.

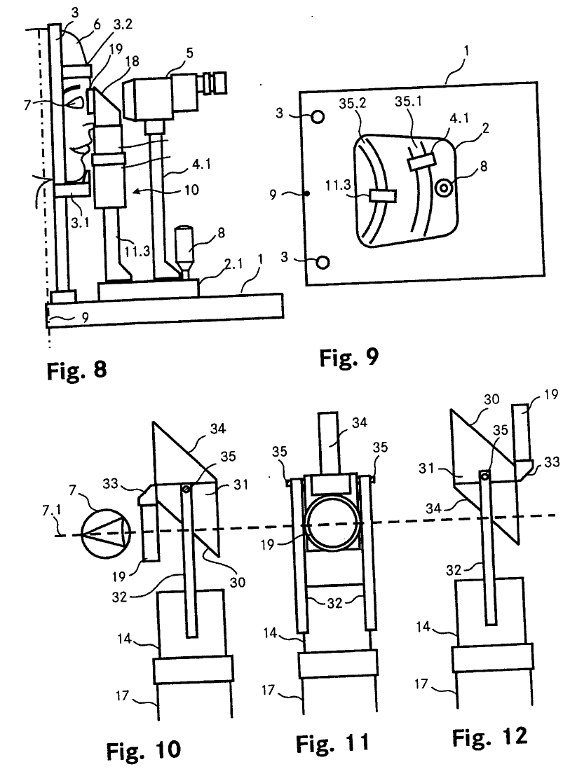

FIG. 8 shows a further example for embodying the invention. The slit-lamp unit illustrated is of the same design as the unit illustrated in FIG. 1, except for the holders for the microscope 5 and the illuminating means 10. In FIG. 9, the table 1 with the cross-slide 2.1 is illustrated, in turn, from above.

Here, the microscope 5 is not connected to the cross-slide 2.1 via an L-shaped arm, but via a vertically arranged elongated microscope arm 4.1. The microscope arm 4.1 is connected, in turn, to the cross-slide 2.1 via a rail 35.1, which is of circular design and is located on the top side of the cross-slide 2.1.

The holder 11 of the illuminating means 10 is also designed in a similar way. The carrier 11.3, on which the prism 18, the image recording device 14 and the light source 17 are fastened, is connected to the cross-slide 2.1 via a rail 35.2. This rail 35.2 is also of circular design and is located on the top side of the cross-slide 2.1. Both the microscope arm 4.1 and the carrier 11.3 can be swiveled circularly along the rails 35.1 and 35.2, respectively, by means of a roller or slide bearing. In so doing, both of them describe a circular movement about a virtual axis of rotation 9.1.

In order to examine the anterior of the eye (not illustrated), the cross-slide 2.1 together with the microscope 5 and illuminating means 10 is positioned in front of the eye at the required distance with the aid of the guide lever 8. The radii of the rails 35.1, 35.2 are selected in this case in such a way that the virtual axis of rotation 9.1 runs in the region of the eye 7 for this position of the cross-slide 2.1. By swiveling the microscope 5 or the illuminating means 10, it is therefore possible to view or illuminate the surface of the eye 7 from various angles.

If, as illustrated in FIG. 8, the aim is to examine the fundus of the eye 7, the cross-slide 2.1 together with the microscope 5 and illuminating means 10 can be displaced to be closer to the eye 7 with the aid of the guide lever 8 until the prism 18 with the lens 19 is finally positioned directly in front of the eye 7. It is not possible with conventional slit-lamp units, since the axis of rotation about which the microscope 5 can be rotated is implemented as a mechanical axis or shaft, and the latter collide, upon displacement of the cross-slide towards the eye 7, with the body of the patient before the prism 18 is sufficiently close to the eye 7. Such a collision is avoided in the inventive device, since the axis of rotation 9.1 is implemented by the circular slides 35.1, 35.2 and is therefore present only virtually but not in reality.

Illustrated in FIGS. 10 to 12 is a further variant of the design of the deflecting device in the case of which the deflecting device is implemented as a mirror 30. The mirror is fastened on a holder 31 which is connected to the image recording device 14 via a linkage 32. The linkage 32 keeps the holder at a specific distance above the image recording device 14. The light emerging from the image recording device 14 for illuminating the fundus of the eye 7 is reflected onto the eye 7 from the mirror 30 through the lens 19. Conversely, the light emerging from the eye 7 passes the lens 19 and is deflected onto the image recording device 14 by the mirror 30. The lens 19 is held in front of the mirror 30 by a lens holder 33, the lens holder 33 itself being fastened on the holder 31. FIG. 10 shows the deflecting device from the side, FIG. 11 from the front, that is to say seen from the eye 7.

In order to achieve as good as image of the fundus as possible, the mirror 30 must in this case be at least as wide as the diameter of the lens 19. This is best seen from FIG. 11.

Such a wide mirror 30 would, however, have a disturbing effect if the anterior of the eye is to be viewed in a conventional manner with the aid of the microscope 5. Specifically, it would block the view from the microscope 5 onto the eye 7. In order to be able to use this slit-lamp unit to examine the anterior of the eye, as well, it is therefore necessary to be able to replace the wide mirror 30 by a narrower mirror.

The deflecting device illustrated in FIGS. 10 to 12 permits this in the simplest way. Specifically, a narrow mirror 34 is fastened on the top side of the holder 31, the holder 31 being connected to the linkage 32 in such a way that it can be rotated about the horizontal axis of rotation 35, which is supported as appropriate in the upper region of the linkage 32. That is to say, depending on the examination desired, either the wide mirror 30 together with the lens 19, or only the narrow mirror 34 can be introduced into the beam path by simply rotating the holder 31. The deflecting device can also have means for exact positioning, in order to cause the holder 31 to latch into the desired positions.

FIG. 12 shows the deflecting device in the position in which the narrow mirror 34 is located in the illuminating beam path. Here, the eye 7 can be examined in a known way with the aid of the microscope 5. That is to say, the mirror 34 is located directly in front of the microscope 5, the respective beams for stereoscopic examination respectively passing on both sides of the narrow mirror 34.

In order to replace the wide mirror 30 by a narrow mirror 34 (or vice versa), the illuminating means 10 could also be of modular design. Thus, for example, the linkage 32 could be designed in such a way that the wide mirror 30 can be exchanged alone or together with the linkage 32. If the deflecting device does not comprise a mirror, but a prism, it is, of course, also possible simply to mount another, narrower prism on the image recording device.

Of course, the image recording device 14 can also be of modular design such that, depending on the application, it is possible to use other image recording devices 14, for example in the form of different image adapters for analog and digital images or for photographs or video sequences. For example, it would also be possible to use an image adapter for generating stereoscopic images, which uses, for example, different components of the total viewing beam, that is to say different or else partially overlapping regions of the CCD chip 25, in order to generate stereo images of the fundus of the eye which could then be viewed stereoscopically on an appropriately equipped monitor.

It may be stated in summary that the invention permits a single slit-lamp unit to be used to examine both front and rear eye sections with a quality not achieved to date, and to display corresponding images on a monitor. Ease of operation is in no way to be diminished in this case. If, for example, a changeover is to be made from examining the foreground to examining the background, the deflecting device can be positioned directly in front of the eye with a few manipulations, the desired illumination can be selected and the narrow mirror can be replaced by a broad one.

1. Device for viewing an eye (7) of a patient, in particular a slit-lamp unit, comprising viewing means (5) for viewing a front section of the eye and illuminating means for illuminating the front section of the eye, characterized in that the illuminating means are designed in such a way that it is also possible to illuminate a rear section of the eye, and that the device comprises optical imaging means for imaging the rear eye section, and an image acquisition device for acquiring images of the eye with the aid of an image recording device, the image recording device being designed and capable of being positioned such that it is possible thereby to image the rear eye section without reflections ophthalmoscopically.

2. Device according to claim 1, characterized in that the illuminating means comprise a light source for generating an illuminating beam, and can be switched over from background illumination to foreground illumination.

3. Device according to claim 1 or 2, characterized in that the image acquisition device comprises a deflecting device, it being possible to position the deflecting device in front of the eye such that a viewing beam emerging from the eye can be deflected through the image recording device.

4. Device according to one of claims 1 to 3, characterized in that the illuminating means comprise a light source and form a unit with the image acquisition device, it being possible to deflect a viewing beam generated by the light source onto the eye by the deflecting device.

5. Device according to claim 4, characterized in that the illuminating beam can be deflected onto the eye at a variable angle of incidence.

6. Device according to claim 4 or 5, characterized in that the illuminating beam comprises a plurality of annularly arranged individual beams.

7. Device according to one of claims 1 to 6, characterized in that the image acquisition device comprises a holder with a base and a carrier, the image recording device being fastened on the carrier, the base being connected moveably to the device, and the holder having guide means for displacing the carrier relative to the base.

8. Device according to claim 7, characterized in that the guide means are of rail-type design, and the carrier can be displaced with the image recording device in a translatory fashion along the base in the direction of the eye.

9. Device according to one of claims 1 to 8, characterized in that the image acquisition device comprises an image displaying device for displaying the images acquired by the image acquisition device.

10. Device according to claim 9, characterized in that the image displaying device comprises a photographic camera, a video camera or a CCD camera.

11. Device according to one of claims 1 to 10, characterized in that the optical imaging means for imaging the rear eye section are positioned near the eye.

12. Device according to one of claims 1 to 11, characterized in that the image acquisition device is designed in such a way that it can be switched over between at least two magnifications.
